(19) United States
(12) Patent Application Publication
(10) Pub. No.: US 2025/0303105 A1
(43) Pub. Date: Oct. 2, 2025

(54) FINGER-WEARABLE ANTI-STRESS DEVICE

(71) Applicant: Christian Davide VESCOVI, Caravaggio (Bergamo) (IT)

(72) Inventors: Nicola D'ALESSANDRO, Cologno Monzese (Milano) (IT); Fabrizio MOTTA, Carenno (Lecco) (IT)

(21) Appl. No.: 18/864,600

(22) PCT Filed: May 4, 2023
(86) PCT No.: PCT/IB2023/054628

§ 371 Date: Nov. 11, 2024

(30) Foreign Application Priority Data
May 10, 2022 (IT) 102022000009605

(51) Int. Cl. A61M 21/02; A61M 21/00; G06M 1/10; G06M 1/272

(52) U.S. Cl. CPC A61M 21/02; G06M 1/108; G06M 1/272; A61M 2021/005; A61M 2205/50; A61M 2205/584; A61M 2205/587; A61M 2205/8206; A61M 2209/088

(57) ABSTRACT

A finger-wearable anti-stress device having a ring and a main body is provided. The ring extends along and about a rotation axis and delimits a finger seat suitable for housing at least partially a finger of a user. A ring seat is provided in the main body, in which the ring is rotationally engaged with respect to the main body to allow rotation of the main body with respect to the ring and about the rotation axis due to a torque imposed by the finger of the user on the ring. The finger-wearable anti-stress device has a revolution counter sensor configured to count a number of complete relative rotations between the ring and the main body.

### FIELD OF APPLICATION

The present invention relates to a finger anti-stress device. In particular, the anti-stress device that is an object of the present invention is wearable on a finger of a user.

The finger anti-stress device according to the present invention is in the field of objects for play or stress relief.

The anti-stress device of the present invention, in particular, is suitable to be used in a wide range of applications, from the children's toys field, to the automotive gadget field, or as a keychain.

### PRIOR ART

There are, in the art, anti-stress devices that may be used with the fingers. For example, some anti-stress devices in the prior art are shaped to be held by two fingers of one hand, and to be brought to move, for example rotationally, by exerting a push with the fingers of the other hand.

Disadvantageously, such known anti-stress devices are not suitable for being used with a single hand, and require both hands to be free to use them, that is, they force the user to devote both hands to their use.

Further, these devices are limited to being used for anti-stress purposes and do not provide an indication on the frequency of their use.

### DISCLOSURE OF THE INVENTION

Therefore, there appears to be a significant need to provide a finger anti-stress device capable of overcoming the drawbacks typical of the state of the art.

Thus, the present invention is intended to overcome the drawbacks of finger anti-stress devices of the prior art by providing a finger anti-stress device that may be used with a single hand, is adaptable to a wide range of functions, and in particular is suitable for providing an indication of its use.

This need is satisfied by an anti-stress device according to the independent claim 1. The dependent claims describe preferred or advantageous embodiments of the invention, comprising further advantageous features.

### DESCRIPTION OF THE DRAWINGS

The features and advantages of the finger anti-stress device will become apparent from the description below of a number of preferred embodiments, given by way of non-limiting example, with reference to the attached figures, wherein:

FIG. 1a and 1b each show a perspective view of an anti-stress device according to one embodiment of the present invention, each from a different point of view;

FIG. 2 shows the anti-stress device of FIG. 1, in a plan view;

FIG. 3 shows a sectional view of the anti-stress device of FIG. 2, sectioned along the plane A-A shown in FIG. 2;

FIG. 4 shows a detailed view of the detail B of FIG. 3;

FIG. 5 shows a perspective view of a ring for an anti-stress device according to an embodiment of the present invention;

FIG. 6 shows a further perspective view of the ring of FIG. 5;

FIG. 7 shows an elevation view of the ring of FIG. 5;

FIG. 8 shows a schematic perspective view of a printed circuit board according to an embodiment of the present invention;

FIGS. 9a and 9b each show a perspective view of a portion of the shell respectively, according to an embodiment of the present invention.

### DETAILED DESCRIPTION

With reference to the aforesaid figures, a finger anti-stress device, in particular wearable on a finger, has been indicated as a whole with the reference number 1. For clarity and ease of description, in the remainder of this discussion the finger-wearable anti-stress device will be referred to in short as an anti-stress device.

According to the present invention, the anti-stress device 1 comprises a ring 3 and a main body 10.

The ring 3 extends along and about a rotation axis Y and delimits a finger seat 300 suitable for housing at least partially a user's finger.

In the present discussion, the term “ring” refers without limitation to an object having a substantially annular body, which preferably is substantially symmetrical and hollow in the center, with either a totally open or a blind or partially blind cavity.

A ring seat 23 is obtained in the main body 10, in which the ring 3 is rotationally engaged with respect to the main body 10, to allow free rotation of the main body 10 with respect to the ring 3 and about said rotation axis Y due to a torque imposed by the user's finger on the ring 3.

Preferably, the ring seat 23 is a through cavity obtained in the main body 10.

In one embodiment, the ring 3 is engaged in the ring seat 23 by means of magnetic coupling.

The anti-stress device 1 further comprises a power supply device 90, such as a battery, and a revolution counter sensor 40, which is supplied by said power supply device 90. The revolution counter sensor 40 is integral in rotation with the main body 10 or with the ring 3 and is configured to count the number of complete relative rotations between the ring 3 and the main body 10.

It is clear that the term “counting” means, without limitation, that the revolution counter sensor 40 is configured to generate a complete rotation signal at each complete relative rotation between the ring 3 and the main body 10.

Preferably, the revolution counter sensor 40 comprises an electronic unit (such as an electronic circuit or a logic unit) configured to receive the complete rotation signal generated by the revolution counter sensor 40.

In one embodiment, the electronic unit is configured to process the complete rotation signal to generate an increment of a certain numerical value of revolutions preferably stored in a storage unit.

In a second embodiment, the electronic control unit is configured to process the complete rotation signal to transform this complete rotation signal into an activation command for a viewing system or an auxiliary notification system (e.g., auditory or visual), described later in this discussion with reference to a preferred embodiment of the invention.

That is, in this embodiment, the complete rotation signal is directly transformed into an activation command without numerically increasing a numerical value of revolutions.

The power supply device 90 is housed in the main body 10 and/or on the ring 3.

In other words, when a user inserts his/her finger into the finger seat 300 and exerts a rotary thrust force on the anti-stress device 1 with the same finger inserted into the finger seat 300, the main body 10 rotates about the ring 3.

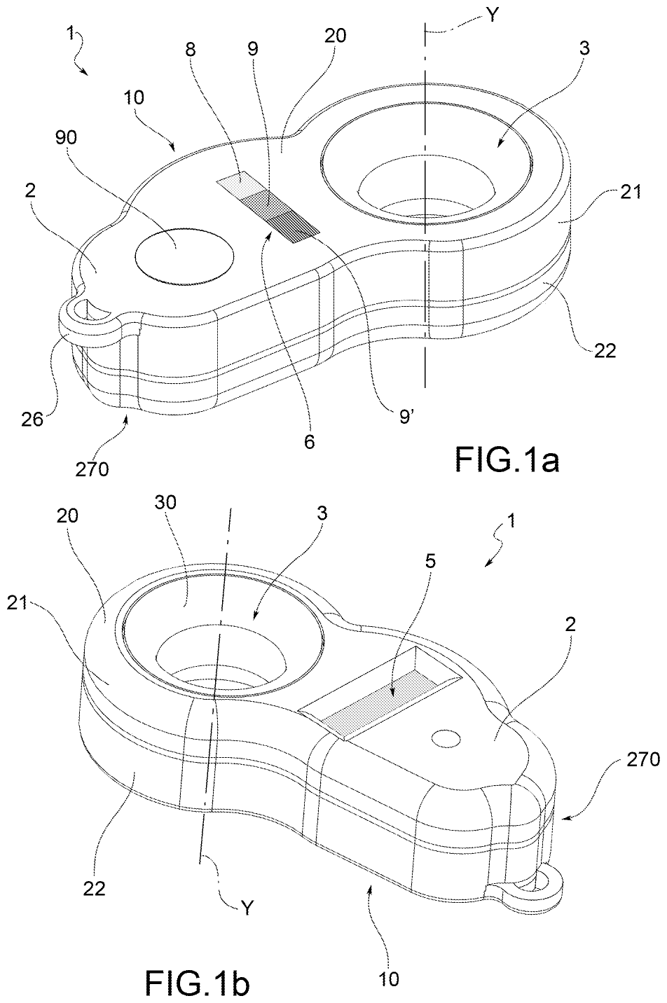

According to one embodiment, for example as shown in FIG. 1a, the power supply device 90 comprises a battery housed in the main body 10.

According to one embodiment, the power supply device comprises a system for converting mechanical power into electric power. For example, this conversion system comprises a dynamo, configured to convert a relative rotary motion between the ring 3 and the main body 10 into electric power. According to one embodiment, the conversion system is directly connected for supplying the revolution counter sensor 40. According to one variant, the conversion system is connected to a battery so that the generated electric power recharges the battery.

It is clear to the person skilled in the art that when the conversion system comprises a dynamo, it is possible for the conversion system, and thus the power device, to be housed partially on the ring 3 and partially in the main body, for example, the magnets of the dynamo on the ring 3 and the windings on the main body, or vice versa.

In other words, the conversion system makes it possible to obtain an anti-stress device 1 that is self-powering or self-charging due to the same rotary motion.

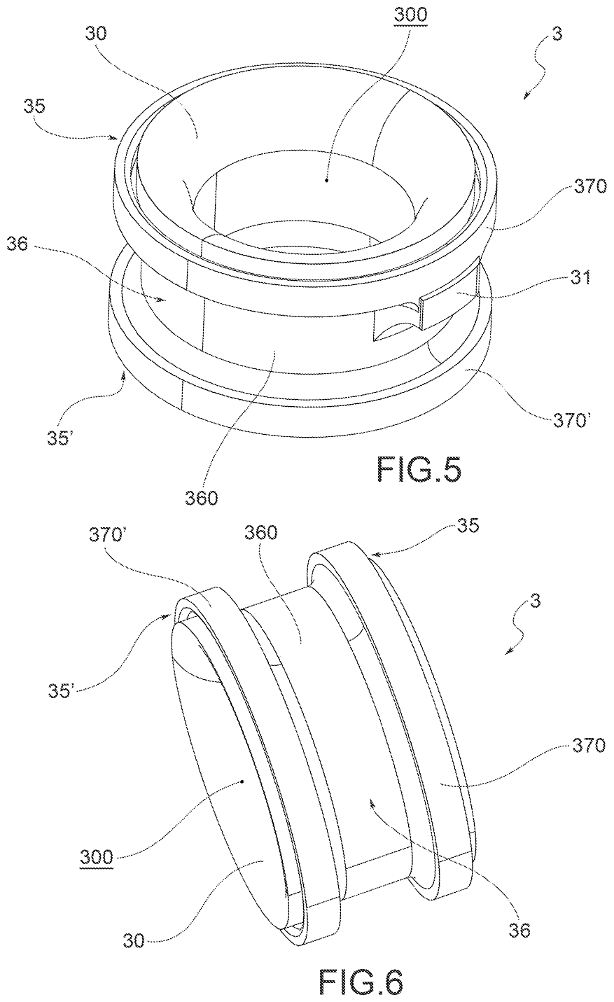

Preferably, with reference to the example embodiment shown in FIGS. 5 to 7, the ring m is composed of a cylindrical body.

In one embodiment, the finger seat 300 is preferably a through cavity of the ring 3.

In an alternative embodiment, the finger seat 300 is a blind cavity of the ring 3, i.e., the finger seat 300 of the ring 3 is a cavity closed on one side.

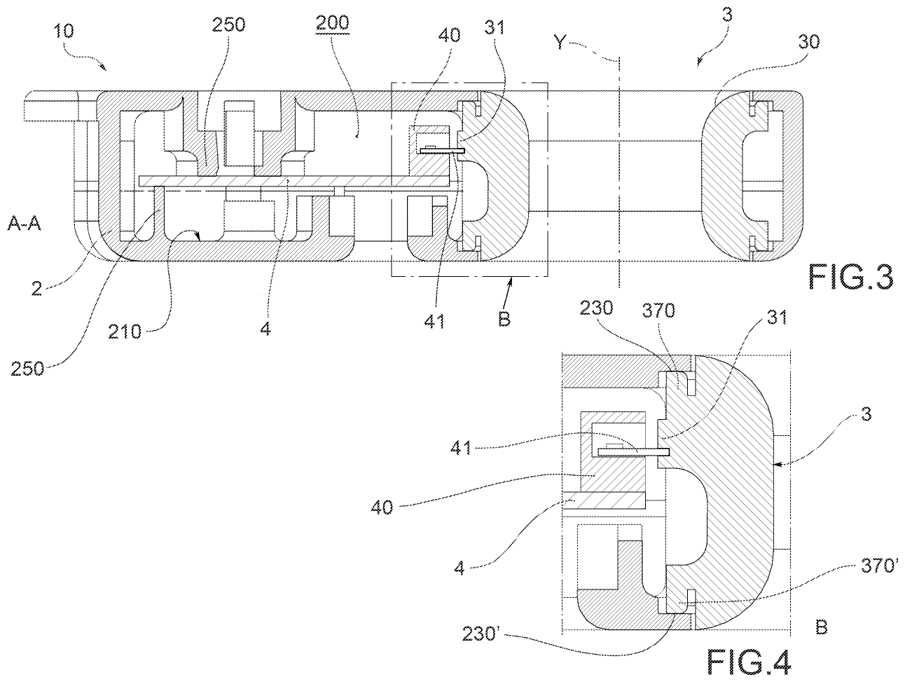

In a preferred embodiment, for example shown in FIGS. 3 and 4, the revolution counter sensor 40 is contained in the main body 10 and integral therewith.

In an alternative embodiment, the revolution counter sensor 40 is placed on the ring 3 and is integral therewith.

In a preferred embodiment, the anti-stress device 1 comprises a counting lever 41 operatively connected to the revolution counter sensor 40.

According to this embodiment, the ring 3 comprises an activation tooth 31 that projects outwards therefrom, i.e., on the opposite side with respect to the finger seat 300, so that, when the user imposes a torque on the ring 3 with a finger, operating the rotation of the main body 10 about said ring 3, the activation tooth 31 is suitable for triggering the counting lever 41 at every rotation, so that the revolution counter sensor 40 is configured to increase the count of the rotation for every activation of the counting lever 41.

In a particularly advantageous embodiment, the counting lever 41 is shaped to be activated only when the rotation occurs in one direction about the rotation axis Y, and not in both directions of rotation.

Preferably, with reference to the example in FIG. 8, the counting lever 41 is equipped with a ramp surface 401, suitable for entering in contact with the activation tooth 31 during the relative rotations between the main body 10 and the ring 3.

Further, preferably, the counting lever 41 is elastically engaged with the revolution counter sensor 40, so that the activation tooth 31, when engaged with the ramp surface 401, activates the counting lever 41 with each rotation by pressing it to switch from a protruding configuration to a pressed configuration. Further, this counting lever 41 is suitable for returning configuration due to the elastic to the protruding engagement with the revolution counter sensor 40 once the activation tooth 31 is no longer in a position to activate the counting lever 41.

In a preferred embodiment, through the elastic engagement and shape of the counting lever 41, the counting may be activated only in one direction of rotation.

In this preferred embodiment, with reference to the example in FIG. 8, the counting lever 41 preferably comprises an abutment surface 402, incident to the ramp surface 401.

The abutment surface 402 is a curved surface, or a linear surface, suitable for abutting with the activation tooth 31.

In particular, when the counting lever 41 is in a protruding configuration, if the rotation of the main body 10 about the ring 3 occurs in a first direction, the activation tooth 31 is suitable for compressing the ramp surface 401, pressing the lever and triggering the counting of the revolution counter sensor 40. If, on the other hand, the rotation of the main body 10 about the ring 3 occurs in a second direction, opposite to the first direction, the activation tooth 31 is suitable for abutting with the abutment surface 402, and not allowing the compression of the ramp surface 401.

In this embodiment, the count of revolution is only allowed in one direction of rotation, clockwise or counterclockwise, depending on how the counting lever 41 is positioned.

In an alternative embodiment, revolution counting is allowed in both directions of rotation.

According to this embodiment of the invention, the revolution counter sensor 40 comprises an electronic encoder, preferably an angle encoder, suitable for detecting the relative rotations between the ring 3 and the main body 10.

Preferably, in such an embodiment, the ring 3 does not comprise any activation teeth 31.

In an advantageous variant embodiment, for example shown in FIG. from 5 to 7, the ring 3 extends along and about the rotation axis Y between an upper end 350 and a lower end 350′ and comprises an upper enlarged portion 35 at said upper end 350 and a lower enlarged portion 35′ at said lower end 350′.

Further, in this variant embodiment, the ring 3 comprises a narrow central portion 36 between the upper enlarged portion 35 and the lower enlarged portion 35′.

The upper and lower enlarged portions 35, 35′ extend radially with respect to the rotation axis Y by a greater thickness than the narrow central portion 36.

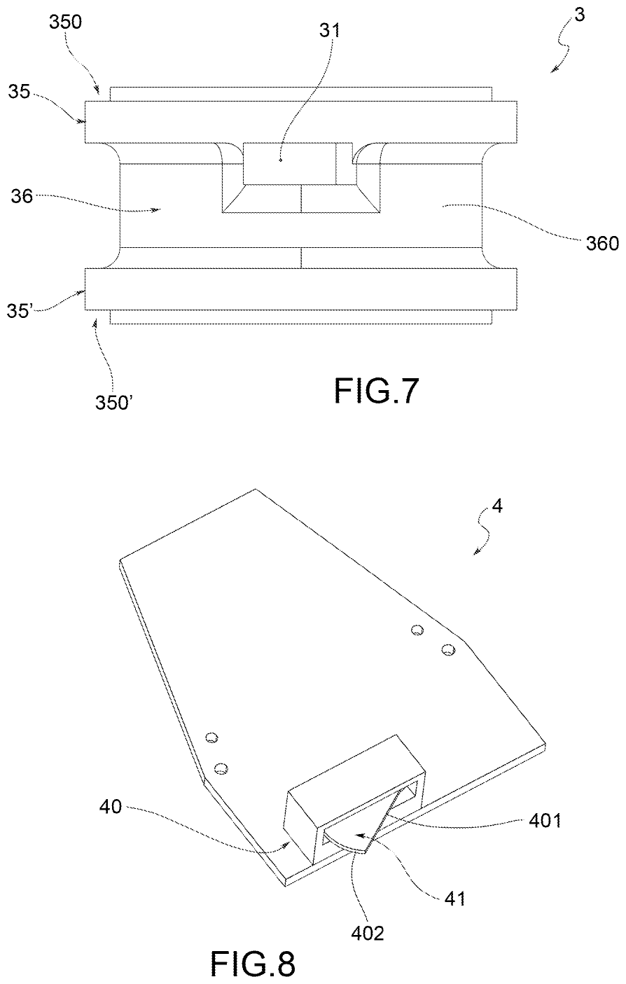

Preferably, as visible in the example embodiment in FIG. 7, the activation tooth 31 protrudes externally from a side wall 360 of said narrow central portion 36.

Also, preferably, the tooth 31 is made in one piece with the ring 3. That is, the tooth 31 is obtained in one piece, integrally with the ring 3.

In one embodiment of the invention, with reference to the embodiment example in FIGS. 5 and 6, the upper enlarged portion 35 and the lower enlarged portion 35′ each comprise a respective annular relief 370, 370′.

In this embodiment, and as shown in the example in FIG. 3, and better depicted in the enlargement in FIG. 4, the ring seat 23 is delimited by an upper seat edge 230 and a lower seat edge 230′, suitable for abutting with the respective annular relief 370, 370′ of the ring 3 for blocking the axial sliding of the ring 3 in the ring seat 23 along the rotation axis Y.

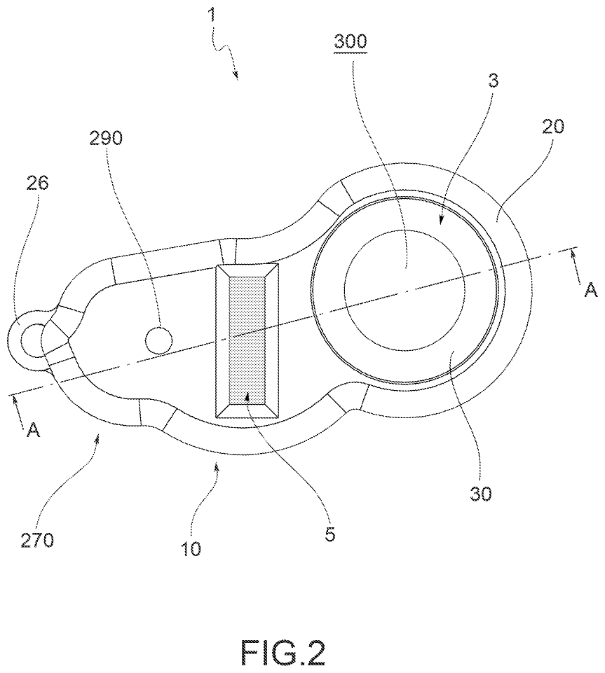

According to a preferred variant embodiment, and as visible in the example embodiment in FIG. 2, moreover, the anti-stress device comprises a viewing system 5, preferably a display, operatively connected to the revolution counter sensor 40, and suitable for showing digitally the number of rotations counted by the revolution counter sensor 40.

In a preferred embodiment, further, the main body 10 comprises a hooking means 26 suitable for hooking one or more keys.

Advantageously, this embodiment allows the anti-stress device 1 to be used simultaneously as a keychain and as a toy. Advantageously, in fact, the main body 10 is rotatable about the ring 3 even when the keys are hooked to the hooking means 26.

In a preferred embodiment, and as visible in the example embodiment in FIG. 1a the anti-stress device 1 comprises an auxiliary viewing system 6, preferably an LED operatively connected to the revolution counter sensor 40. This auxiliary viewing system 6 is configured to provide a graphic representation of the stress level of the user as a function of the number of rotations counted by the revolution counter sensor 40.

In a preferred embodiment, the anti-stress device 1 comprises both the viewing system 5 and the auxiliary viewing system 6.

Preferably, the viewing system 5 and auxiliary viewing system 6 are placed on the same side of the anti-stress device 1. That is, the user sees both viewing systems from a single perspective.

Alternatively, and with reference to the example embodiment shown in FIGS. 1a and 1b, the viewing system 5 and the auxiliary viewing system 6 are positioned on opposite sides of the anti-stress device 1. That is, the user sees the viewing system 5 from a first perspective, and the auxiliary viewing system 6 from a second perspective. In other words, to switch from seeing the viewing system 5 to the auxiliary viewing system 6, the user has to turn over the anti-stress device 1.

The auxiliary viewing system 6 is configured to show a first coloring 8 when the number of rotations counted by the revolution counter sensor 40 is less than a certain predetermined threshold value, and to show a second coloring 9 when the number of rotations counted by the revolution counter sensor 40 is higher than said predetermined threshold value.

Preferably, moreover, with reference to FIG. 1a, the auxiliary viewing system 6 is configured to show a first coloring 8, a second coloring 9, and a third coloring 9′, depending on the number of rotations counted by the revolution counter sensor 4.

Further, preferably, the revolution counter sensor 40 is configured to be automatically reset, preferably every 24 hours.

Alternatively, or additionally, the revolution counter sensor 40 is configured to be manually reset by the user according to his will.

Preferably, when the revolution counter sensor 40 is configured to be automatically reset, for example, the auxiliary viewing system 6 is a colored bar configured to light up according to the rotation number signal detected by the revolution counter sensor 40, for example, showing the colors green, yellow, and red where each color indicates a certain stress level. For example, when the number of rotations in a day exceeds a certain upper threshold value, the colored bar lights up according to said number of rotations detected by the revolution counter sensor 40 to show the color red, indicating a high level of stress. Conversely, when the number of rotations in a day does not reach a certain lower threshold value, the colored bar lights up according to said number of rotations detected by the revolution counter sensor 40 to show the color green, indicating a low stress level.

Preferably, when the number of rotations in a day is between the lower threshold value and the upper threshold value, the bar lights up according to said number of rotations detected by the revolution counter sensor 40 to show the color yellow, indicating an intermediate stress level.

In one embodiment, the main body 10 extends about the ring 3. In other words, the entire circumference of the ring 3 is at least partially surrounded by the main body 10.

In still other words, the side wall 360 of the ring 3 always faces along the circumference at least partially toward the main body 10.

In one embodiment, the ring 3 always remains bound to the main body 10. Preferably, the ring 3 may not be separated from the main body 10 during rotation. That is, the ring 3 remains bound to the main body 10 even during rotation.

Advantageously, this variant avoids the loss of the components of the anti-stress device while preventing possible damage due to separation of the ring from the main body during rotation.

In a preferred embodiment, the main body 10 comprises a shell 2.

In one embodiment, the shell 2 contains a printed circuit board 4 operatively connected to the revolution counter sensor 40.

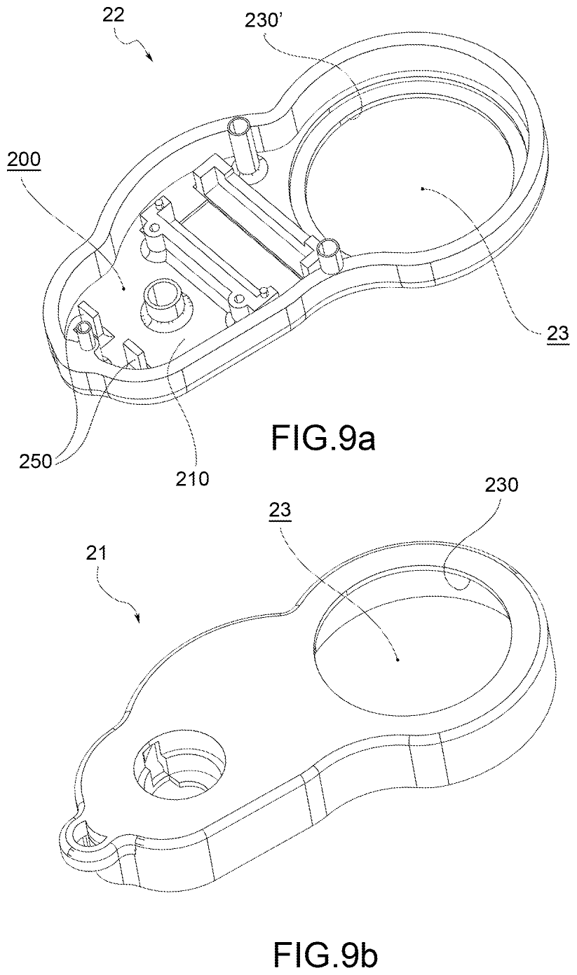

In an advantageous variant, with reference to the example embodiment shown in FIGS. 1a and 1b in assembly, and in FIGS. 9a and 9b in individual pieces, the shell 2 consists of a first shell portion 21 and a second shell portion 22, assembled together.

In this variant, preferably, the ring seat 23 is partially obtained in the first shell portion 21 and partially in the second shell portion 22. In other words, the ring 3 engaged in the ring seat 23 is partially engaged with the first shell portion 21 and with the second shell portion 22.

Preferably, the first shell portion 21 and the second shell portion 22 are glued together, are attached to each other by interlocking.

In an advantageous embodiment, the shell 2 and the ring 3 are made entirely of hard plastic.

Preferably, with reference to the example in FIGS. 3 and 4, the shell 2 comprises an outer wall 20 that delimits an inner cavity 200 in which the printed circuit board 4 is contained.

Further, preferably, the outer wall 20 of the shell 2 comprises an inner shell surface 210 having one or more locking protuberances 250 that constrain the printed circuit board 4 in the inner cavity 200, so that the printed circuit board 4 is locked within said inner cavity 200, even during the rotation of the main body 10 about the ring 3. In other words, these locking protuberances 250 keep the circuit board 4 in the same position, i.e., keeping it fixed, during the rotation of the main body 10 about the ring 3.

In the configuration wherein the revolution counter sensor 40 is configured to be reset, the printed circuit board 4 comprises a reset command, and on the body 2 an auxiliary opening 290 is obtained, suitable for allowing access to this reset command from the printed circuit board 4.

In a preferred embodiment, the shell 2 fully surrounds the ring 3 in the ring seat 23.

Further, with reference to FIG. 1a and 1b, in a preferred embodiment, the shell 2 comprises an elongated portion 270 that projects laterally with respect to the ring seat 23, i.e., the ring seat 23 is obtained in the main body 10 on the opposite side with respect to this elongated portion 270.

Advantageously, this preferred embodiment allows the user to effectively implement the rotation of the main body 10 with respect to the ring 3, also acting as a mass and generating a flywheel effect to facilitate rotation without requiring the application of high torque.

Preferably, the ring 3 is located at one end of the main body 10. In other words, preferably, the ring 3 is positioned away from the center of the main body 10.

That is, preferably, defining a barycenter of the anti-stress device 1, this barycenter is not located on the rotation axis Y. In other words, preferably, the ring 3 is offset from the center of gravity of the anti-stress device 1.

Advantageously, this allows the anti-stress device to be conveniently operated with one hand and to impart less torque on the ring to keep the main body rotating. This also further increased the flywheel effect that facilitates the rotation of the device.

Preferably, moreover, when the anti-stress device 1 is also configured to serve as a keychain, the hooking means 26 protrudes from the main body 10 at said elongated portion 270 of the body 2.

Further, preferably, the hooking means 26 is in one piece with the shell 2.

This configuration further increases the flywheel effect, which is greatly accentuated by the presence of keys hanging from said hooking means 26.

Preferably, there is a gap between the ring 3 and the ring seat 23.

Even more preferably, this gap is suitable to be at least partially filled with a lubricant to facilitate the relative rotation between said ring 3 and the main body 10.

Advantageously, therefore, the relative rotation between the main body 10 and the ring 3 is allowed without the need for intermediate bearings. It is clear that the person skilled in the art may still provide for intermediate bearings to allow for the relative rotation between the parts without departing from the scope of protection of the present invention.

In a preferred embodiment, the upper enlarged portion 35 and the lower enlarged portion 35′ of the ring 3 comprise a curved surface 30, which is seamlessly connected to the outer wall 20 of the shell 2.

Advantageously, this shape, in addition to giving an aesthetically pleasing appearance to the product, provides greater ergonomics thereto, making it more practical to use.

Preferably, moreover, the ring has a smooth inner surface, free of roughness, so that a finger may be inserted into the finger seat 300 without causing discomfort to the user.

Preferably, the shell 2 features a design or is covered with a printed film on which a decorative motif is depicted.

In a preferred embodiment, the anti-stress device 1 further comprises light-signaling means, such as LEDs or laser emitters, installed on the ring 3 or the main body 10, suitable for emitting light during rotations of the main body 10 with respect to the ring 3.

That is, in a preferred embodiment, the anti-stress device 1 is equipped with additional lights, which generate light games, either intermittently, or continuously, or with color changes, depending on the user's rotational actuation of the main body 10 with respect to the ring 3.

In a variant embodiment, the anti-stress device 1 comprises additional sensors, installed on the ring 3 or on the main body 10, suitable for detecting vital parameters of the individual who is using the device, such as respiratory rate, heart rate, blood saturation, and blood pressure.

For example, the additional sensors comprise a heart rate monitor and/or a pressure sensor, and/or a sensor configured to detect the saturation level.

Innovatively, the finger-wearable anti-stress device according to the present invention makes it possible to resolve all the drawbacks of the anti-stress devices of the prior art.

Advantageously, the anti-stress device according to the present invention is of simple design, comprising a limited number of components.

Further, advantageously, the anti-stress device according to the present invention is usable by one hand only, and thus allows the user to have the other hand free to perform other activities.

According to a further advantage, the anti-stress device according to the present invention allows for tracking the use of the anti-stress device through the use of a revolution counter sensor.

Further, advantageously, the count operated by the revolution counter sensor is effectively shown to the user, for example, digitally, by means of a display with numbers, and/or graphically, for example, by means of an LED strip or colored bar.

Advantageously, further, the anti-stress device lends itself to a wide range of applications, such as a keychain, through the hooking means.

Advantageously, further, the anti-stress device is ergonomic, due to the shape that connects the ring and the main body in continuity, and is convenient to use.

It is clear that, to the embodiments of the aforesaid anti-stress device, a person skilled in the art, in order to meet specific needs, could make variations or substitutions of elements with functionally equivalent ones. Such variants are also included within the scope of protection as defined by the following claims. Moreover, each variant described as belonging to a possible embodiment may be implemented independently of the other variants described.

### LIST OF REFERENCE NUMBERS

1 anti-stress device
10 main body
Y rotation axis
2 shell
20 outer wall
21 first shell portion
22 second shell portion
23 ring seat
26 hooking means
200 inner cavity
210 inner shell surface
230 upper seat edge
230′ lower seat edge
250 locking protuberances
270 elongated portion
290 auxiliary opening
3 ring
30 curved surface
31 activation tooth
33 relief
35 upper enlarged portion
35′ lower enlarged portion
36 narrow central portion
300 finger seat
350 upper end
350′ lower end
360 side wall
370, 370′ annular relief
4 printed circuit board
40 revolution counter sensor
41 counting lever
401 ramp surface
402 abutment surface
5 viewing system
6 auxiliary viewing system
8 first coloring
9 second coloring
9′ third coloring
90 power supply device

1. A finger-wearable anti-stress device, comprising a ring and a main body,
wherein the ring extends along and about a rotation axis and delimits a finger seat suitable for housing at least partially a finger of a user,
wherein a ring seat is obtained on provided in the main body, in which the ring is rotationally engaged with respect to the main body to allow a rotation of the main body with respect to the ring and about said rotation axis due to a torque imposed by the finger of the user on the ring,
said finger-wearable anti-stress device further comprising a power supply device housed in the main body and/or on the ring, and a revolution counter sensor, supplied by said power supply device and rotationally integral with the main body or the ring, said revolution counter sensor being configured to count a number of complete relative rotations between the ring and the main body.

2. The finger-wearable anti-stress device of claim 1, wherein the revolution counter sensor is contained in the main body and is integral therewith.

3. The finger-wearable anti-stress device of claim 2, further comprising a counting lever operatively connected to the revolution counter sensor, and wherein the ring comprises an activation tooth which projects outwards and integrally therewith, on an opposite side with respect to the finger seat, so that, when the user imposes the torque on the ring with the finger, operating the rotation of the main body about said ring, said activation tooth is suitable for triggering said counting lever at every rotation, so that the revolution counter sensor is configured to increase the count of the rotations for every activation of the counting lever.

4. The finger-wearable anti-stress device of claim 1, wherein the ring extends along and about the rotation axis between an upper end and a lower end, and comprises an upper enlarged portion at said upper end, a lower enlarged portion at said lower end and comprises a narrow central portion between said upper enlarged portion and said lower enlarged portion, and wherein the upper and lower enlarged portions extend radially with respect to the rotation axis over a greater thickness than the narrow central portion.

5. The finger-wearable anti-stress device of claims 3, wherein the ring extends along and about the rotation axis between an upper end and a lower end, and comprises an upper enlarged portion at said upper end, a lower enlarged portion at said lower end and a narrow central portion between said upper enlarged portion and said lower enlarged portion, wherein the upper and lower enlarged portions extend radially with respect to the rotation axis over a greater thickness than the narrow central portion, and wherein the activation tooth projects outwards from a side wall of said narrow central portion.

6. The finger-wearable anti-stress device of claim 4, wherein the upper enlarged portion and the lower enlarged portion each comprise a respective annular relief, and wherein the ring seat is delimited by an upper seat edge and by a lower seat edge, said upper and lower seat edges being suitable for abutting with a respective annular relief of the ring for locking an axial sliding of the ring in the ring seat along the rotation axis.

7. The finger-wearable anti-stress device of claim 1, further comprising a viewing system, optionally a display, operatively connected to the revolution counter sensor, and suitable for showing digitally the number of the complete relative rotations counted by the revolution counter sensor.

8. The finger-wearable anti-stress device of claim 1, wherein the main body comprises a hooking means suitable for hooking one or more keys.

9. The finger-wearable anti-stress device of claim 1, further comprising an auxiliary viewing system, the auxiliary viewing system optionally being a light-emitting diode (LED) strip, operatively connected to the revolution counter sensor, said auxiliary viewing system being configured to provide a graphic representation of a stress level of the user as a function of the number of the complete relative rotations counted by the revolution counter sensor.

10. The finger-wearable anti-stress device of claim 9, wherein the auxiliary viewing system is configured to show a first coloring when the number of the complete relative rotations counted by the revolution counter sensor is less than a certain predetermined threshold value, and to show a second coloring when the number of the complete relative rotations counted by the revolution counter sensor is higher than said predetermined threshold value.

11. The finger-wearable anti-stress device of claim 1, wherein the main body comprises a shell containing a printed circuit operatively connected to the revolution counter sensor.

12. The finger-wearable anti-stress device of claim 11, wherein the shell consists of a first shell portion and a second shell portion, assembled together, and wherein the ring seat is partially obtained in the first shell portion and partially in the second shell portion, so that the ring engaged in said ring seat is partially engaged with the first shell portion and the second shell portion.

13. The finger-wearable anti-stress device of claim 11, wherein the shell comprises an outer wall delimiting an inner cavity, and wherein in which the printed circuit is contained in the inner cavity.

14. The finger-wearable anti-stress device of claim 11, wherein the shell totally surrounds the ring in the ring seat and comprises an elongated portion projecting laterally with respect to said seat, i.e., the ring seat is obtained being provided in the main body on an opposite side with respect to said elongated portion.

15. The finger-wearable anti-stress device of claim 1, wherein between the ring and the ring seat there is a gap for facilitating the relative rotations between the ring and the main body.

16. The finger-wearable anti-stress device of claims 4, wherein the main body comprises a shell containing a printed circuit operatively connected to the revolution counter sensor, wherein the shell comprises an outer wall delimiting an inner cavity, wherein the printed circuit is contained in the inner cavity, and wherein the upper enlarged portion and the lower enlarged portion of the ring comprise a curved surface seamlessly connected to the outer wall of the shell.

17. The finger-wearable anti-stress device of claim 1, wherein the ring is positioned at one end of the main body.

18. The finger-wearable anti-stress device of claim 1, wherein the power supply device comprises a system for converting mechanical power into electric power, suitable for converting a relative rotary motion between the ring and the main body into electric power for supplying the revolution counter sensor.

19. The finger-wearable anti-stress device of claim 1, wherein the power supply device is a battery.
